(19) United States
(12) Patent Application Publication
(10) Pub. No.: US 2008/0058391 A1
(43) Pub. Date: Mar. 6, 2008

(54) NOVEL COMPOUNDS

(75) Inventors: Christopher Norbert Johnson, Harlow (GB); David Timothy MacPherson, Harlow (GB); Giancarlo Trani, Harlow (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, Greenford (GB)

(21) Appl. No.: 11/835,522

(22) Filed: Aug. 8, 2007

(30) Foreign Application Priority Data
Aug. 10, 2006 (GB) 0615943.8
Jul. 13, 2007 (GB) 0713681.5

(51) Int. Cl. A61K 31/44; A61P 1/00; A61P 25/00; C07D 213/02

(52) U.S. Cl. 514/357; 546/337

(57) ABSTRACT

Compounds of formula (I) and pharmaceutically acceptable salts thereof: 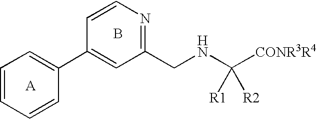
wherein; each of R¹ to R⁴ is independently selected from hydrogen and C₁₋₄ alkyl and each of rings A and B independently is optionally further substituted by up to three substituents, each of which is independently selected from the group consisting of halogen, hydroxy, C₁₋₄alkoxy, C₁₋₄alkyl, C₁₋₅alkanoyl, CF₃, CF₃O and cyano; with the proviso that ring A must contain at least one CF₃ group, are useful in the treatment of diseases and conditions mediated by modulation of use-dependent voltage-gated sodium channels.

### BACKGROUND OF THE INVENTION

The present invention relates to novel compounds, salts thereof, and to the use thereof in treating diseases and conditions mediated by modulation of voltage-gated sodium channels. In addition, the invention relates to compositions containing compounds of the invention and processes for their preparation.

Voltage-gated sodium channels are responsible for the initial phase of the action potential, which is a wave of electrical depolarisation usually initiated at the soma of the neuron and propagated along the nerve axon to the terminals. At the terminals, the action potential triggers the influx of calcium and the release of neurotransmitter. Drugs, such as lidocaine, that block voltage-gated sodium channels are used as local anaesthetics. Other sodium channel blockers, such as lamotrigine and carbamazepine are used to treat epilepsy. In the latter case, partial inhibition of voltage-gated sodium channels reduces neuronal excitability and reduces seizure propagation. In the case of local anaesthetics, regional block of sodium channels on sensory neurons prevents the conduction of painful stimuli. A key feature of these drugs is their use-dependent mechanism of action. The drugs are thought to stabilise an inactivated configuration of the channel that is adopted rapidly after the channel opens. This inactivated state provides a refractory period before the channel returns to its resting (open) state ready to be reactivated. As a result, use-dependent sodium channel blockers retard the firing of neurons at high frequency, for example in response to painful stimuli, and will help to prevent repetitive firing during periods of prolonged neuronal depolarisation that might occur, for example, during a seizure. Action potentials triggered at low frequencies, for example in the heart, will not be significantly affected by these drugs, although the safety margin differs in each case, since at high enough concentrations each of these drugs is capable of blocking the resting or open states of the channels.

The voltage-gated sodium channel family is made up of four brain specific subtypes, NaV1.1, 1.2, 1.3 and 1.6; as well as NaV1.4, which is found only on skeletal muscle; NaV1.5, which is specific to cardiac muscle; and NaV1.7, 1.8, 1.9, which are found predominantly on sensory neurons. The hypothesised binding site for use-dependent sodium channel blockers is highly conserved between all the subtypes. As a result, drugs such as lidocaine, lamotrigine and carbamazepine do not distinguish between then. However, selectivity is achieved as a result of the different frequencies at which the channels normally operate.

Drugs that block voltage-gated sodium channels in a use-dependent manner are also used in the treatment of certain psychiatric disorders.

WO 99/26614 describes certain N² substituted 2-methyl alaninamides, which act as sodium channel blockers, as well as their use in methods for the treatment of neuronal damage following global and focal ischaemia, and for the treatment, prevention or amelioration of pain, as anticonvulsants, as antimanic depressants, as local anaesthetics, as antiarrhythmics and for the treatment or prevention of diabetic neuropathy. The compounds described in WO 99/26614 include 2-[3-(4-fluorophenoxy)-5-pyridylmethylamino]-2-methyl-propanamide (also known as 2-methyl-N²-({3-[4-fluorophenoxy]-5-pyridinyl}methyl)alaninamide).

WO99/35125 describes the use of alpha-aminoamide derivatives as analgesic agents. The compounds described in WO99/35125 include ralfinamide which is the compound of formula: 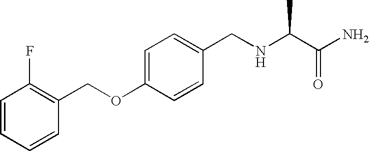
Ralfinamide is in clinical development for the treatment of neuropathic pain.

WO92/01675 describes certain bis-aryl compounds for use in the treatment of hypersensitive and inflammatory conditions including rheumatoid arthritis, gout, psoriasis and inflammatory bowel disease.

### DESCRIPTION OF THE INVENTION

The object of the present invention is to identify a compound which blocks voltage-gated sodium channels in a use-dependent manner.

According to a first aspect, the invention provides the compound of formula (I) or a pharmaceutically acceptable salt thereof: 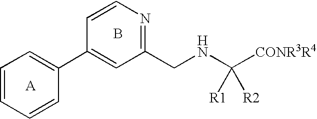
wherein; each of R¹ to R⁴ is independently selected from hydrogen and C₁₋₄alkyl, and each of rings A and B independently is optionally further substituted by up to three substituents, each of which is independently selected from the group consisting of halogen, hydroxy, C₁₋₄alkoxy, C₁₋₄alkyl, C₁₋₅alkanoyl, CF₃, CF₃O and cyano, with the proviso that ring A must contain at least one CF₃ group.

As used herein halogen means fluorine, chlorine, bromine or iodine and any alkyl, alkoxy or alkanoyl group may be straight or branched chain.

In certain embodiments of the invention, R¹ and R² are methyl, and/or ring B is not further substituted, and/or ring A is substituted only by a single trifluoromethyl group, and/or R³ and R⁴ are hydrogen.

Compounds of the invention include the following:
2-Methyl-N²-({4-[4-(trifluoromethyl)phenyl]-2-pyridinyl}methyl)alaninamide
N¹,2-Dimethyl-N²-({4-[4-(trifluoromethyl)phenyl]-2-pyridinyl}methyl)alaninamide
N¹,N¹,2-Trimethyl-N²-({4-[4-(trifluoromethyl)phenyl]-2-pyridinyl}methyl)alaninamide
N²-({4-[4-(Trifluoromethyl)phenyl]-2-pyridinyl}methyl)glycinamide
N²-({4-[4-(Trifluoromethyl)phenyl]-2-pyridinyl}methyl)-L-alaninamide
N²-({4-[4-(4-Trifluoromethyl)phenyl]-2-pyridinyl}methyl)-D-alaninamide
2-Methyl-N²-({4-[2-(trifluoromethyl)phenyl]-2-pyridinyl}methyl)alaninamide
2-Methyl-N²-({4-[3-(trifluoromethyl)phenyl]-2-pyridinyl}methyl)alaninamide

and pharmaceutically acceptable salts thereof.

It will be appreciated by the person skilled in the art that the compound of formula (I) may have, at least one chiral centre and will therefore exist in stereoisomeric forms (e.g. diastereoisomers and enantiomers) and the invention extends to each of these stereoisomeric forms and to mixtures thereof including racemates.

The different stereoisomeric forms of the compound of formula (I) may be obtained according to methods well known in the literature, for example by separation one from the other by the usual methods such as preparative HPLC or by chromatographic purifications. A racemic mixture may either be separated using preparative HPLC and a column with a chiral stationary phase or resolved to yield individual enantiomers utilising methods known to those skilled in the art. Any given isomer may also be obtained by stereospecific or asymmetric synthesis. In addition, chiral intermediate compounds may be resolved and used to prepare individual stereoisomeric forms of chiral compounds of the invention.

The invention also extends to any tautomeric forms and mixtures thereof.

The compound of formula (I) may form pharmaceutically acceptable salts. The pharmaceutically acceptable salts are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al., J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

It will be appreciated by those skilled in the art that certain protected derivatives of the compound of formula (I), which may be made prior to a final deprotection stage, may not possess pharmacological activity as such, but may, in certain instances, be administered orally or parenterally and thereafter metabolised in the body to form the compound of the invention which is pharmacologically active. Such derivatives may therefore be described as “prodrugs”. All protected derivatives and prodrugs of the compound of formula (I) are included within the scope of the invention. Examples of suitable pro-drugs for the compound of formula (I) are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference).

Hereinafter, the compound of formula (I), its pharmaceutically acceptable salts, and its prodrugs, defined in any aspect of the invention (except intermediate compounds in chemical processes) are referred to as “the compounds of the invention”.

The compounds of the invention include pharmaceutically acceptable solvates such as hydrates. Also included within the scope of the compounds of the invention are polymorphs thereof.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulfur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

As discussed hereinabove, it is believed that compounds of the invention are particularly useful for the treatment of diseases and conditions mediated by modulation of voltage-gated sodium channels, and can be used in the prevention and alleviation of pain including visceral pain and neuropathic pain.

Therefore, according to a further aspect, the invention provides compounds of the invention for use as a medicament, such as a human medicament.

According to a further aspect the invention provides the use of compounds of the invention in the manufacture of a medicament for treating or preventing a disease or condition mediated by modulation of voltage-gated sodium channels.

In one embodiment, compounds of the invention may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis); musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of the invention may also be useful in the amelioration of inflammatory disorders, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel-syndrome, inflammatory bowel disease, gastroesophageal reflux disease); other conditions with an inflammatory component such as migraine, multiple sclerosis, myocardial ischemia.

Compounds of the invention are also believed to be useful in the treatment and/or prevention of disorders treatable and/or preventable with anti-convulsive agents, such as epilepsy including post-traumatic epilepsy, obsessive compulsive disorders (OCD), sleep disorders (including circadian rhythm disorders, insomnia & narcolepsy), tics (e.g. Giles de la Tourette's syndrome), ataxias, muscular rigidity (spasticity), and temporomandibular joint dysfunction.

Compounds of the invention may also be useful in the treatment of bladder hyperrelexia following bladder inflammation.

Compounds of the invention may also be useful in the treatment of neurodegenerative diseases and neurodegeneration such as dementia, particularly degenerative dementia (including senile dementia, Alzheimer's disease, Pick's disease, Huntington's chorea, Parkinson's disease and Creutzfeldt-Jakob disease, motor neuron disease); The compounds may also be useful for the treatment of amyotrophic lateral sclerosis (ALS) and neuroinflamation.

Compounds of the invention may also be useful in neuroprotection and in the treatment of neurodegeneration following stroke, cardiac arrest, pulmonary bypass, traumatic brain injury, spinal cord injury or the like.

Compounds of the invention may also be useful in the treatment of tinnitus, and as local anaesthetics.

In a further embodiment, diseases or conditions that may be mediated by modulation of voltage-gated sodium channels are selected from the list consisting of [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes with Delusions and With Hallucinations; Substance-Induced Psychotic Disorder including the subtypes With Delusions (293.81) and With Hallucinations (293.82); and Psychotic Disorder Not Otherwise Specified (298.9); and

ii) Depression and mood disorders for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90).

iii) Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00):

iv) Substance-related disorders including Substance Use Disorders such as Substance Dependence, Substance Craving and Substance Abuse; Substance-Induced Disorders such as Substance Intoxication, Substance Withdrawal, Substance-induced Delirium, Substance-Induced Persisting Dementia, Substance-Induced Persisting Amnestic Disorder, Substance-Induced Psychotic Disorder, Substance-induced Mood Disorder, Substance-induced Anxiety Disorder, Substance-Induced Sexual Dysfunction, Substance-induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-induced Persisting Amnestic Disorder, Alcohol-Induced Psychotic Disorder, Alcohol-induced Mood Disorder, Alcohol-Induced Anxiety Disorder, Alcohol-induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

v) Enhancement of cognition including the treatment of cognition impairment in other diseases such as schizophrenia, bipolar disorder, depression, other psychiatric disorders and psychotic conditions associated with cognitive impairment, e.g. Alzheimer's disease:

vi) Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46) Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersonmnia Type, Parasomnia Type and Mixed Type; sleep apnea and jet-lag syndrome:

vii) Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder, Binge Eating Disorder; and Eating Disorder Not Otherwise Specified (307.50):

viii) Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism).

ix) Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):

x) Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9): and

xi) Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

In a further embodiment, diseases or conditions that may be mediated by modulation of voltage gated sodium channels are Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)).

It will be appreciated that references herein to “treatment” extend to prophylaxis, prevention of recurrence and suppression or amelioration of symptoms (whether mild, moderate or severe) as well as the treatment of established conditions. The compound of the invention may be administered as the raw chemical but the active ingredient is preferably presented as a pharmaceutical formulation.

According to a further aspect, the invention provides a pharmaceutical composition comprising a compound of the invention, in association with one or more pharmaceutically acceptable carrier(s), diluents(s) and/or excipient(s). The carrier, diluent and/or excipient must be “acceptable” in the sense of being compatible with the other Ingredients of the composition and not deleterious to the recipient thereof.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration or via inhalation to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams, gels or lotions, eye ointments and eye or ear drops, impregnated dressings or adhesive patches, and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulfate. The tablets may be coated according to methods well known in normal pharmaceutical practice. Oral liquid preparations may be in the form of, for example aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For compositions suitable and/or adapted for inhaled administration, it is preferred that the compound is in a particle-size-reduced form, and more preferably the size-reduced form is obtained or obtainable by micronisation. The preferable particle size of the size-reduced (e.g. micronised) compound or salt or solvate is defined by a D50 value of about 0.5 to about 10 microns (for example as measured using laser diffraction).

Aerosol compositions, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, the pharmaceutical composition may comprise a dry powder inhalable composition. Such a composition can comprise a powder base such as lactose, glucose, trehalose, mannitol or starch, the active compound (preferably in particle-size-reduced form, e.g. in micronised form), and optionally a performance modifier such as L-leucine or another amino acid, cellobiose octaacetate and/or metals salts of stearic acid such as magnesium or calcium stearate. Preferably, the dry powder inhalable composition comprises a dry powder blend of lactose and the active compound. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. Most importantly, it is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 J D Zwolle, Netherlands).

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose of e.g. the dry powder composition can be administered by inhalation via the device such as the DISKUS™ device, marketed by GlaxoSmithKline. The DISKUS™ inhalation device is for example described in GB 2242134 A, and in such a device at least one container for the pharmaceutical composition in powder form (the container or containers preferably being a plurality of sealed dose containers mounted longitudinally in a strip or ribbon) is defined between two members peelably secured to one another; the device comprises: a means of defining an opening station for the said container or containers; a means for peeling the members apart at the opening station to open the container; and an outlet, communicating with the opened container, through which a user can inhale the pharmaceutical composition in powder form from the opened container.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter-sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit may contain from 50-500 mg of the active ingredient. The dosage as employed for adult human treatment may range from 10 to 3000 mg per day, depending on the route and frequency of administration. Such a dosage corresponds to 0.1 to 50 mg/kg per day.

For the treatment of visceral pain or IBS, a compound of the invention may typically be administered twice daily via the oral route, with each dose containing between 15 and 325 mg of the active compound.

For the treatment of neuropathic pain, a compound of the invention may typically be administered, twice daily via the oral route, with each dose containing between 35 and 230 mg of the active compound.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

It will be appreciated that the invention includes the following further aspects. The preferred embodiments described for the first aspect extend these further aspects. The preferred disease and conditions described above extend, where appropriate, to these further aspects.
i) A compound of the invention for use in treating or preventing a disease or condition mediated by modulation of voltage-gated sodium channels.
ii) A method of treatment or prevention of a disease or condition mediated by modulation of voltage-gated sodium channels in a mammal comprising administering an effective amount of a compound of the invention.

The present invention also provides a process for the preparation of a compound of formula (I) or a pharmaceutically acceptable salt thereof, which process comprises:

(a) reaction of a compound of formula (II) 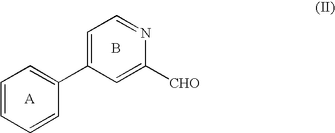
or an optionally protected derivative thereof, with a compound of formula (III) 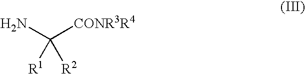
wherein R¹ to R⁴ are as defined above,

(b) deprotecting a compound of formula (I) which is protected;

(c) interconversion to other compounds of formula (I) and/or forming a pharmaceutically acceptable salt and/or solvate;

(d) as appropriate, separation of diastereomeric or enantiomeric mixtures of compounds of formula (I) or protected derivatives thereof.

Reaction of compounds of formula (II) with compounds of formula (III) according to process (a) are typically carried out in the presence of a reducing agent such as sodium cyanoborohydride or sodium triacetoxyborohydride in a suitable solvent such as methanol, ethanol or 1,2-dichloroethane, either at ambient temperature or elevated temperature e.g. reflux, and optionally in the presence of an acid such as acetic acid. In the process (a), the compound of formula (III) may optionally be used in the form of an acid addition salt such as the hydrochloride.

In processes (a), (b) and (c), examples of protecting groups and the means for their removal can be found in T. W. Greene ‘Protective Groups in Organic Synthesis’ (J. Wiley and Sons, 1991). Suitable amine protecting groups include sulfonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid. A further amine protecting group includes methyl which may be removed using standard methods for N-dealkylation (e.g. 1-chloroethyl chloroformate under basic conditions followed by treatment with methanol).

Separations according to process (d) may be carried out using established methodology, e.g. by chromatography, resolution as diastereomeric salts or crystallisation.

Compounds of formula (II) may be prepared by reaction of a compound of formula (IV) with a compound of formula (V) 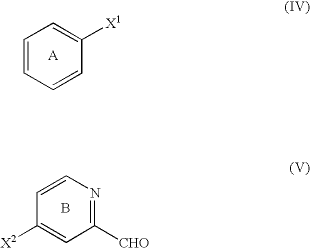
or optionally protected derivatives thereof, wherein X¹ and X² are chosen such that one is a leaving group such as halogen (e.g. bromine or iodine) or trifluoromethylsulfonyloxy and the other is a metal or metalloid residue such as trialkylstannyl or B(OH)₂, using conditions suitable for cross coupling reactions, typically involving use of a transition metal such as palladium in the presence of a suitable ligand such as triphenylphosphine or 1,1′-bis(diphenylphosphino)ferrocene, a base such as sodium carbonate or sodium hydrogen carbonate and an appropriate solvent such as 1,2-dimethoxyethane or a mixture of solvents such as toluene and water, either at ambient temperature or elevated temperature (e.g. reflux) or by use of microwave irradiation.

Alternatively, compounds of formula (I) may be prepared by reaction of a compound of formula (IV) as defined above with a compound of formula (VI)

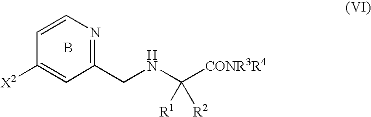

Wherein X² and R¹ to R⁴ are as defined above, using conditions suitable for cross coupling reactions, typically involving use of a transition metal such as palladium in the presence of a suitable ligand such as triphenylphosphine or 1,1′-bis(diphenylphosphino)ferrocene, a base such as sodium carbonate or sodium hydrogen carbonate and an appropriate solvent such as 1,2-dimethoxyethane or a mixture of solvents such as toluene and water, either at ambient temperature or elevated temperature (e.g. reflux) or by use of microwave irradiation.

Compounds of formula (VI) may be prepared by reaction of a compound of formula (III) and a compound of formula (V) as defined above in a manner analogous to that described for process (a) above.

Compounds of formula (III), (IV) and (V) are known in the literature or can be prepared by analogous methods or by methods similar to those described in the descriptions below.

It will be appreciated that compounds of formulas (II), (III), (IV) and (V) may be obtained as mixtures of diastereomers and/or enantiomers. Such mixtures may optionally be separated using established methodology, e.g. by chromatography, resolution as diastereomeric salts or crystallisation.

A method suitable for preparing the compound of Example 1a on a larger scale is outlined in the Scheme below:

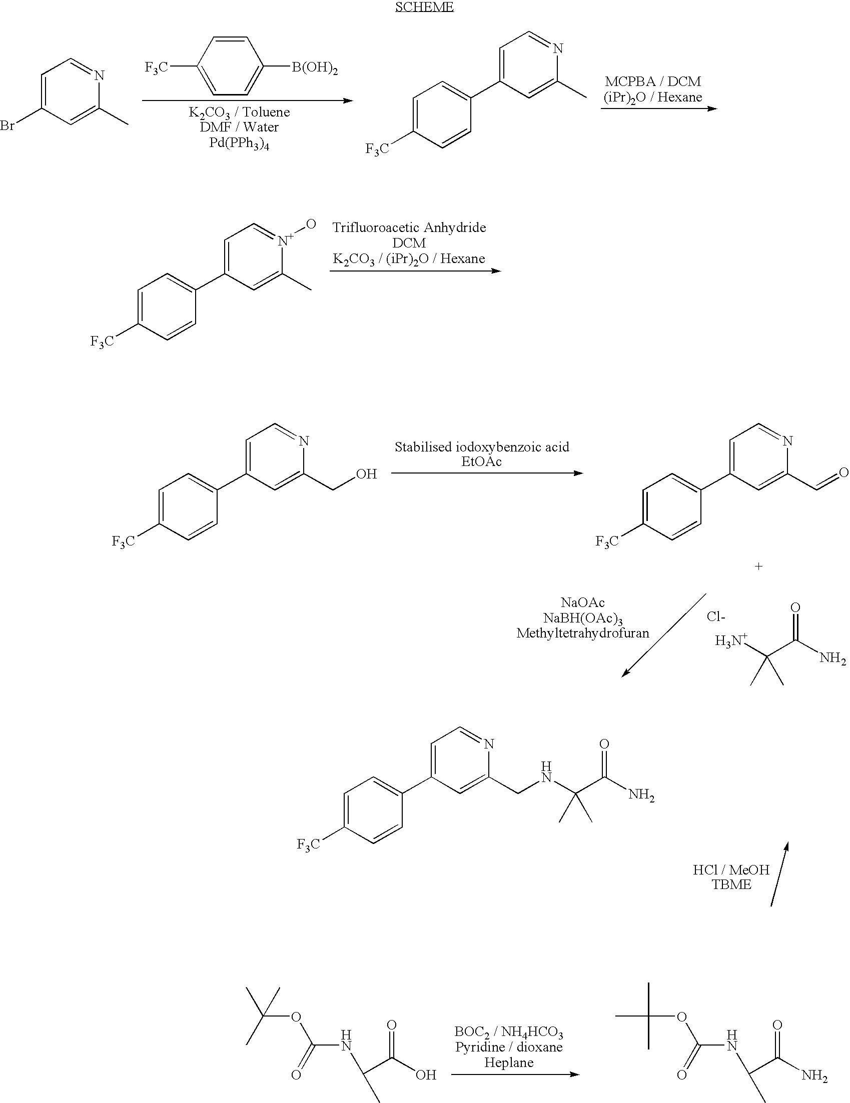

### EXPERIMENTALS

The invention is illustrated by the Examples described below.

In the procedures that follow, after each starting material, reference to a Description is typically provided. This is provided merely for assistance to the skilled chemist. The starting material may not necessarily have been prepared from the batch referred to.

Compounds of the invention are named using ACD/Name PRO 6.02 chemical naming software (Advanced Chemistry Development Inc., Toronto, Ontario, M5H2L3, Canada).

LC/Mass spectra were obtained using one of the following methods:

(A) 5 Minute Method

Agilent 1100 series HPLC system coupled with a Waters ZQ Mass Spectrometer. LC analysis was performed on a Waters Atlantis column (50×4.6 mm, 3 μm) (mobile phase: 97% [water+0.05% HCO₂H]/3% [CH₃CN+0.05% HCO₂H] for 0.1 min, then a gradient to 3% [water+0.05% HCO₂H]/97% [CH₃CN+0.05% HCO₂H] over 3.9 min, and then held under these conditions for 0.8 min): temperature=30° C.; flow rate=3 mL/min: Mass spectra were collected using electrospray and/or APCI. In the mass spectra only one peak in the molecular ion cluster is reported. The UV detection range is from 220 to 330 nm.

(B) 2 Minute Method

Hardware: Waters Acquity Binary solvent Manager, Waters Acquity Sample Manager, Waters Acquity Column Oven, Waters Acquity Photo Diode Array, Waters ZQ Mass Spectrometer, Polymer Labs ELSD PL1000, Computer System. XP SP2 Software: Waters MassLynx v4.1

Column: Acquity UPLC BEH C₁₈ 1.7 μm 2.1 mm×50 mm, column oven set to 40 degrees centigrade

Solvents, A—Aqueous solvent=Water 0.1% Formic Acid+10 mM Ammonium Acetate, B—Organic solvent=MeCN:Water 95:5+0.05% Formic Acid

Instrument settings: Injection volume: 0.5 μl, UV detection: 220 to 330 nm, MS scan range: 100 to 1000 amu, MS scanning rate: 0.2 second scan with a 0.1 second inter scan delay, MS scan function: Electrospray with pos neg switching

Gradient:

| Time | Flow ml/min | % A | % B |
|---|---|---|---|
| 0 | 1 | 97 | 3 |
| 0.1 | 1 | 97 | 3 |
| 1.4 | 1 | 0 | 100 |
| 1.9 | 1 | 0 | 100 |
| 2 | 1 | 97 | 3 |

Proton Magnetic Resonance (NMR) spectra were recorded on a Bruker instrument at 250 or 400 MHz. Chemical shifts are reported in ppm (δ) using tetramethylsilane as internal standard. Splitting patterns are designated as s, singlet; d, doublet; t, triplet; q, quartet; m, multiplet; br, broad. The NMR spectra were recorded at a temperature ranging from 25 to 90° C. When more than one conformer was detected the chemical shifts for the most abundant one are reported.

Chromatography was carried out on silica gel cartridges either on a Flashmaster II (Argonaut) or a Biotage SP4 automated chromatography system and an appropriate elution solvent system.

Mass Directed Automated Preparative (MDAP) HPLC instruments consist of the following: Waters 2525 Binary Gradient Module, Waters 515 Makeup Pump, Waters Pump Control Module, Waters 2767 Inject Collect, Waters Column Fluidics Manager, Waters 2996 Photodiode Array Detector, Waters ZQ Mass Spectrometer, Gilson 202 fraction collector, Gilson Aspec waste collector. Column: Waters Atlantis, dimensions are 19 mm×100 mm (<100 mg scale) and 30 mm×100 mm (>100 mg scale), particle size is 5 μm. Solvents, A: Aqueous solvent=Water+0.1% Formic Acid B: Organic solvent=Acetonitrile+0.1% Formic Acid. Gradients range from 5-30% B in A to 80-99% B in A, depending on HPLC retention time, run time=13.5 minutes. Flow rate=20 ml/min (<100 mg scale), 40 ml/min (>100 mg scale)

The following table lists some abbreviations:

EtOAc Ethyl acetate

DCM Dichloromethane

DMF N,N-dimethylformamide

MeOH Methanol

EDC 1-(3-dimethylaminopropyl)-3-ethylcarbodiimide hydrochloride

HOBT 1-Hydroxybenzotriazole

DMSO Dimethyl sulfoxide

DCE 1,2-Dichloroethane

THF Tetrahydrofuran

Boc [(1,1-Dimethylethyl)oxy]carbonyl

MP-carbonate Macroporous triethylammonium methylpolystyrene carbonate

Pd(dppf)Cl₂ Dichloro[1,1′-bis(diphenylphosphino)ferrocene]palladium

### Description 1

### N²-{[(1,1-Dimethylethyl)oxy]carbonyl}-2-methylalaninamide (D1)

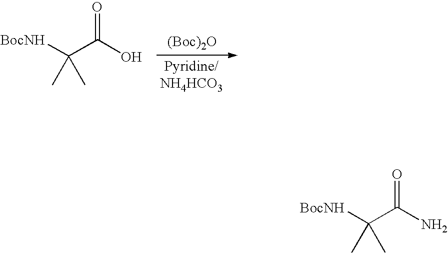

N²-{[(1,1-Dimethylethyl)oxy]carbonyl}-2-methylalanine (8.12 g) and (Boc)₂O (9.603 g) was weighed into a round bottom flask and 1,4-dioxan (200 mL added). Pyridine (2.43 mL) was syringed under argon. NH₄HCO₃ (3.16 g) was added slowly afterwards. Reaction was left overnight over a stirrer hotplate under argon. A small amount of reaction mixture was removed of its solvent using the rotary evaporator and checked by running the NMR and also the NMR of the starting material in DMSO for comparison. The NMR showed that there was still some starting material left in the reaction mixture. Reaction was left for two more days. NMR showed that reaction has gone to completion. The solvent was removed using a rotary evaporator and product was placed in the oven to dry overnight. The title compound D1 was confirmed by NMR.

NMR δ_{H} (CDCl₃) 1.45 (9H, s), 1.51 (6H, s), 4.95 (1H, broad s), 5.40 (1H, broad s), 6.40 (1H, br s).

### Description 1

### Alternative Procedure

### N²-{[1,1-Dimethylethyl)oxy]carbonyl}-2-methylalaninamide (D1)

To a solution of N²-{[1,1-Dimethylethyl)oxy]carbonyl}-2-methylalanine (51.3 g, 0.253 mol), (Boc)₂O (60.6 g, 0.278 mol) and pyridine (21.5 mL) in 1,4-dioxan (1.1 L) under argon was added NH₄HCO₃ (20.4 g, 0.258 mol). The mixture was stirred with a mechanical stirrer under argon for 5 days, the mixture was filtered and the precipitate washed with 1,4-dioxan and the filtrate was concentrated on the rotary evaporator and the resulting solid was dried in the vacuum oven at 40° C. for 2 h to give 50.11 g of white solid (D1), with NMR data consistent with those previously obtained.

### Description 2

### 2-Methylalaninamide hydrochloride (D2)

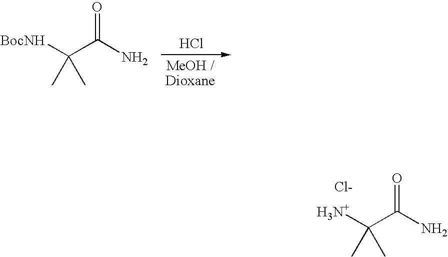

N²-{[(1,1-Dimethylethyl)oxy]carbonyl}-2-methylalaninamide (D1) (8.585 g, 40 mmol) was dissolved in methanol (60 mL) with the aid of sonication and a stirrer hotplate. A solution of HCl in dioxane (4 M, 50 mL) was added and some precipitation was observed after 20 mins. Reaction was left for another 1 hour and 25 mins and was checked using NMR. NMR showed reaction has gone to completion. The reaction mixture was placed on the rotary evaporator to remove the solvent, to give a white powder. The compound was dried in the vacuum oven overnight. The compound was checked using NMR and the spectrum corresponded to the structure of the desired compound (D2).

NMR δ_{H} (DMSO) 1.45 (6H, s), 7.55 (1H, s), 7.79 (1H, s), 8.16 (3H, br s).

### Description 2

### Alternative Procedure

### 2-Methylalaninamide hydrochloride (D2)

N²-{[(1,1-Dimethylethyl)oxy]carbonyl}-2-methylalaninamide (D1) (50.11 g) dissolved in methanol (200 mL)/1,4-dioxan (100 mL) was treated with 4 M HCl in 1,4-dioxan (400 mL) in an ice bath under argon. After 10 minutes the ice bath was removed and the mixture was stirred for 2.5 h under argon at room temperature. The solvents were evaporated on the rotary evaporator and the resulting solid was triturated with diethyl ether (500 mL), filtered and washed with more diethyl ether (2×300 mL) and then dried in the vacuum oven at 35-40° C. for 4 h to give a white solid, 33.63 g (D2), with NMR data consistent with those previously obtained.

### Description 3

### 2-Methylalaninamide (D3)

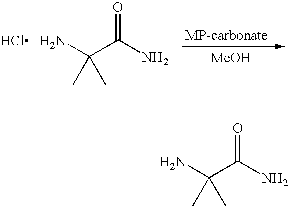

2-Methylalaninamide hydrochloride (D2) (5.44 g, 39.28 mmol) was dissolved in anhydrous MeOH (100 mL). To this solution was added the MP-Carbonate resin (Argonaut) (28.3 g, 78.56 mmol) in one portion and the suspension shaken gently for 4 h. The resin was removed by filtration and washed with MeOH (50 mL). The combined organics were evaporated to afford a brown solid (D3). This was azeotroped with DCE to remove any remaining MeOH.

NMR δ_{H} (MeOD) 1.34 (6H, s).

### Description 4

### (4-Bromo-2-pyridinyl)methanol (D4)

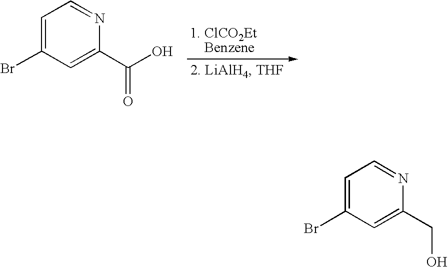

To a suspension of 4-bromo-2-pyridinecarboxylic acid (1.5 g, 7.43 mmol) in benzene (40 mL) was added triethylamine (1.1 mL, 7.8 mmol) and the mixture was sonicated and stirred until it became a clear solution. Ethyl chloroformate (0.743 mL, 7.8 mmol) was added at room temperature and the resulting mixture was stirred for 1/1.5 hours after which it was filtered through a pad of celite, washed with benzene and the filtrate was concentrated to afford a yellow mobile oil. This was then dissolved in dry THF (25 mL) and added dropwise to a suspension of lithium aluminium hydride (7.8 ml, 7.8 mmol+3 mL of dry THF to facilitate the stirring) kept at −78° C. under argon atmosphere. When the addition was complete the bright orange solution was kept stirring at this temperature and followed by TLC after 0.5 hours. It was quenched with a mixture of water/THF (20%) until gas evolution ceased and then allowed to warm to room temperature. The mixture at this point was diluted with EtOAc and water, the phases were separated and the aqueous extracted with EtOAc. The combined organics were washed with saturated aqueous NaHCO₃ and water and then dried over MgSO₄. The crude material (1 g) was purified by flash chromatography (Biotage SP4, 40+S column) with a gradient of EtOAc in hexane to produce the desired product D4 as a yellow oil (700 mg, 50%).

¹H-NMR (CDCl₃): δ 3.39 (1H, broad s), 4.76 (2H, s), 7.39 (1H, m), 7.48 (1H, s), 8.38 (1H, d).

### Description 4

### Alternative Procedure

### (4-Bromo-2-pyridinyl)methanol (D4)

To a stirred suspension of 4-bromo-2-pyridinecarboxylic acid (39.95 g, 0.198 mol) in dry benzene (1.04 L) under argon was added triethylamine (29.0 mL, 0.208 mol) and the mixture was stirred at room temperature for 10 minutes. To the cloudy mixture was added ethyl chloroformate (19.9 mL, 0.208 mol) slowly at room temperature whilst monitoring the reaction temperature (temperature rose from 23° C. to 28° C. during the addition). The resulting mixture was stirred for 1.5 h at room temperature and then filtered through Celite, and the filtrate was concentrated to afford a yellow mobile oil (assumed 0.198 mol for next stage).

To a stirred suspension of lithium aluminium hydride (74 mL of 2 M solution in THF+50 mL of 1 M solution in THF, 0.198 mol) in dry THF (350 mL), under argon at −78° C. was added a solution of the oil from above in dry THF (1.0 L) at such a rate that the internal temperature did not rise above −60° C. When the addition was complete the solution was stirred below −70° C. for 30 minutes. The reaction was quenched by slow addition of 20% water in THF (200 mL), further diluted with THF (500 mL), followed by a saturated solution of Rochelle salt (200 mL) and the mixture was allowed to warm to room temperature. The mixture was filtered through Celite and THF was evaporated from the filtrate. The aqueous concentrate was extracted with ethyl acetate (4×) and the combined extracts were dried and concentrated to give a brown oil. Flash chromatography (silica, elution with 0-80% ethyl acetate in hexane) gave the title compound as a yellow oil (25.43 g) (D4), with NMR data consistent with those previously obtained.

LC-MS: MH+=0.189. C₆H₆BrNO requires 188

### Description 5

### 4-Bromo-2-pyridinecarbaldehyde (D5)

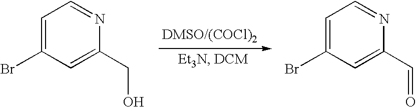

To a solution of the oxalyl chloride (0.364 mL, 4.09 mmol) in dry DCM (30 mL) at −78° C. was added DMSO (0.634 mL, 8.93 mmol) in dry DCM (5 mL) dropwise. The mixture was stirred at this temperature under argon atmosphere for 10 minutes before a solution of (4-bromo-2-pyridinyl)methanol (D4) (700 mg, 3.72 mmol) in dry DCM (15 mL) was added dropwise. After circa 30 minutes, triethylamine (2.6 mL, 18.6 mmol) was added and the cooling bath was removed. The reaction mixture was allowed to stir at room temperature for 1 h after which water was added and it was extracted 3 times with DCM. The combined organics were dried over MgSO₄. The crude material (700 mg) was purified by flash chromatography (Biotage SP4, 25+M column) with a gradient of EtOAc in hexane to afford 425 mg (61%) of the desired product D5.

¹H-NMR (CDCl₃): δ 7.70 (1H, dd), 8.12 (1H, d), 8.61 (1H, d), 10.05 (1H, s)

### Description 5

### Alternative Procedure

### 4-Bromo-2-pyridinecarbaldehyde (D5)

To a solution of oxalyl chloride (13.2 mL, 0.149 mol) in dry dichloromethane (1 L) at −78° C. under argon was added a solution of DMSO (23 mL, 0.324 mol) in dry dichloromethane (180 mL) dropwise over 20 minutes. The mixture was stirred at −78° C. under argon for 15 minutes and then a solution of (4-bromo-2-pyridinyl)methanol (D4) (25.4 g, 0.135 mol) in dry dichloromethane (500 mL) was added dropwise over 30 minutes. The resulting white suspension was stirred at −78° C. for 40-45 minutes and then triethylamine (95 mL, 0.676 mol) was added dropwise over 15 minutes. After stirring at −78° C. for 15 minutes, the mixture was allowed to reach room temperature over ˜1.5 h and then poured into water (400 mL). The organic layer was separated and the aqueous layer was extracted with dichloromethane (3×, ˜1 L total solvent). The combined organic layers were washed with brine (200 mL), dried over MgSO₄ and concentrated. The crude material was purified by flash chromatography on silica gel with a gradient of 0 to 30% ethyl acetate in hexane to afford 20.5 g (82%) of the desired product D5, with NMR data consistent with those previously obtained.

### Description 6

### 4-[4-Trifluoromethyl)phenyl]-2-pyridinecarbaldehyde (D6)

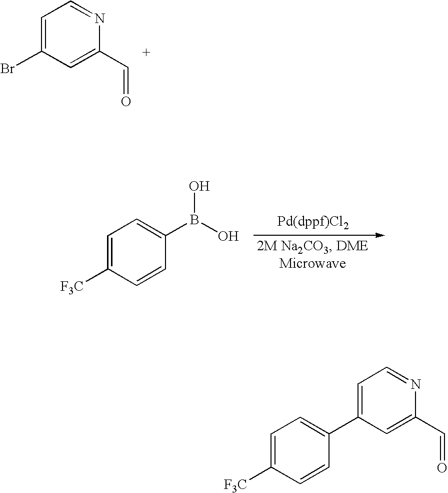

A mixture of 4-trifluoromethylphenylboronic acid (644 mg, 3.39 mmol), 4-bromo-2-pyridinecarbaldehyde (D5) (420 mg, 2.26 mmol) and 2M Na₂CO₃ (4 mL, 7.91 mmol) in dimethoxyethane (12 mL) was degassed for 5-10 minutes in an ultrasonic bath under a flow of argon. Pd(dppf)Cl₂ (92 mg, 0.113 mmol) was added and the resulting mixture was heated with stirring at 130° C. for 10 minutes in a microwave reactor. TLC (EtOAc/hexane 1:1) after 10 minutes showed the reaction went to completion. The mixture was filtered through a pad of celite, washed with EtOAc and concentrated to afford 1 g of crude material which was purified by flash chromatography (Biotage SP4, 40+S column) with a gradient 0 to 50% of EtOAc in hexane to yield 340 mg (60%) of desired product D6.

¹H-NMR (CDCl₃): δ 7.76 (1H, m), 7.81 (4H, s), 8.2 (1H, s), 8.89 (1H, d), 10.18 (1H, s)

### Description 6

### Alternative Procedure

### 4-[4-Trifluoromethyl)phenyl]-2-pyridinecarboxaldehyde. (D6)

A mixture of 4-bromo-2-pyridinecarboxaldehyde (D5) (18.33 g, 98.5 mmol), 4-trifluoromethylphenylboronic acid (20.6 g, 108.4 mmol) and sodium bicarbonate (41.4 g, 492.7 mmol) in toluene (550 mL) and water (55 mL) was degassed with argon for 15 minutes. To this suspension under argon was added tetrakis(triphenylphosphine) palladium(0) (3.42 g, 2.96 mmol) in one portion and the reaction was heated to 90° C. for 18 h. After cooling the solvent was evaporated and the residue suspended in ethyl acetate (1 L). This was filtered and the filter cake washed with ethyl acetate (4×100 mL). The combined organics were evaporated to afford a yellow solid which was purified by flash chromatography (Biotage Flash 75L, silica gel, 3:1→2:1 40-60 petroleum ether/ethyl acetate) to afford the title compound as a yellow solid (22.46 g) (D6), with NMR data consistent with those previously obtained.

LC-MS: [MH⁺]=252, C₁₃H₈F₃NO requires 251.

### Description 7

### N²-{[(1,1-Dimethylethyl)oxy]carbonyl}-N¹,2-dimethylalaninamide (D7)

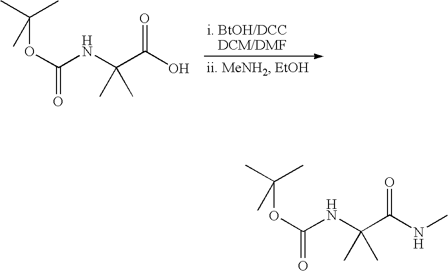

To a solution of N-{[(1,1-dimethylethyl)oxy]carbonyl}-2-methylalanine (3 g, 14.8 mmol) in DCM/DMF (4:1, 45 mL) was added hydroxybenzotriazole (2.2 g, 16.28 mmol) and dicyclohexyl carbodiimide (3.17 g, 15.84 mmol) and the mixture was stirred at room temperature for 30 minutes. A solution of methylamine in ethanol (33% w/w, 2.4 mL, 19.24 mmol) was then added and the progress of the reaction was checked by TLC.

The reaction mixture was filtered to remove insoluble impurities and the filtrate was diluted with DCM (100 mL), washed with saturated aqueous NaHCO₃ (2×50 mL), water, citric acid (10% w/v, 2×50 mL) and brine. The organic layer was dried over MgSO₄ and concentrated in vacuo to afford 1.5 g of crude material. This was purified by flash chromatography (Biotage SP4, 40+S silica cartridge) with a gradient of 0 to 100% of EtOAc in hexane to yield 1.31 g of the title compound (D7).

NMR δ_{H} (CDCl₃): 1.44 (9H, s), 1.48 (6H, s), 2.82 (3H, d, J=4.8 Hz), 4.9 (1H, broad s), 6.5 (1H, broad s).

### Description 8

### N²-{[(1,1-Dimethylethyl)oxy]carbonyl}-N¹,N¹,2-trimethylalaninamide (D8)

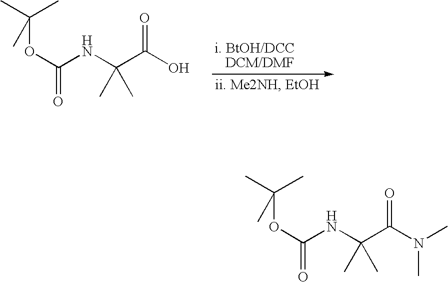

Title compound D8 was prepared via a procedure similar to that described in Description 7 starting from N-{[(1,1-dimethylethyl)oxy]carbonyl}-2-methylalanine (3 g, 14.8 mmol) and using a solution of dimethylamine in ethanol (33% w/w, 3.45 mL, 19.24 mmol). After purification 2.35 g of the title compound D8 was isolated.

NMR δ_{H} (CDCl₃): 1.43 (9H, s), 1.52 (6H, broad s), 3.07 (6H, broad s), 5.1 (1H, broad s).

### Description 9

### N¹,2-Dimethylalaninamide hydrochloride (D9)

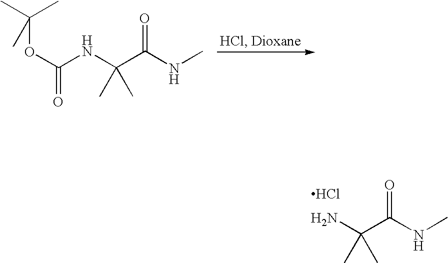

A solution of N²-{[(1,1-dimethylethyl)oxy]carbonyl}-N¹,2-dimethylalaninamide (D7) (1.31 g, 6.06 mmol) In 4 M HCl in dioxane (15 mL) was stirred at room temperature and the progress of the reaction was followed by ¹H-NMR. After 4 h the mixture was concentrated to dryness to yield 915 mg of the title compound D9.

NMR δ_{H} (D₆-DMSO): 1.46 (6H, s), 2.67 (3H, s), 8.28 (4H, m).

### Description 10

### N¹,N¹,2-Trimethylalaninamide hydrochloride (D10)

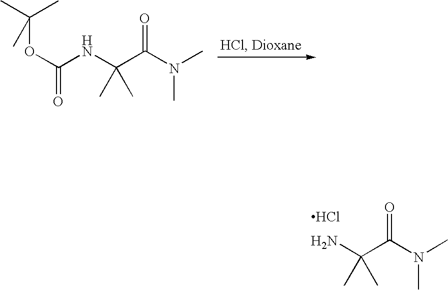

A solution of N²-{[(1,1-dimethylethyl)oxy]carbonyl}N¹,N¹,2-trimethylalaninamide (D8) (2.35 g, 10.2 mmol) in 4 M HCl in dioxane (15 mL) was stirred at room temperature and the reaction was checked by ¹H-NMR. After 4 h the mixture was concentrated to dryness to yield 1.68 g of the title compound D10.

NMR δ_{H} (D₆-DMSO): 1.57 (6H, s), 2.99 (6H, broad s), 8.23 (3H, broad s).

### Description 11

### N²-[(4-Bromo-2-pyridinyl)methyl]-2-methylalaninamide (D11)

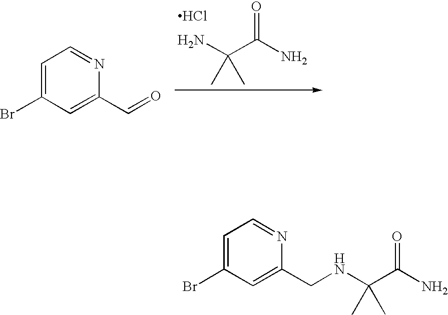

To a solution of 4-bromo-2-pyridinecarbaldehyde (4 g, 21.5 mmol) (D5) in DCE (160 mL) was added 2-methylalaninamide hydrochloride (D2) (4.47 g, 32.25 mmol), NaOAc (2.65 g, 32.25 mmol) and 4 Å molecular sieves (activated in the vacuum oven at 70° C. for 1 day, 20 g) and the resulting mixture was stirred under argon at room temperature. The imine formation was checked by ¹H-NMR and after 18 h, NaBH(OAc)₃ (6.84 g, 32.25 mmol) and acetic acid (1.94 mL, 32.25 mmol) were added. After stirring for 6 h a NMR sample showed reduction of the imine; saturated aqueous sodium bicarbonate solution (110 mL) was added slowly and the solution was stirred at room temperature for 1 hour after which it was filtered through a pad of Celite, washed with DCM (100 mL) and the organic layer was separated. The aqueous phase was extracted with DCM (50 mL) and the combined organics were washed with brine (50 mL), dried over MgSO₄ and concentrated to afford 5.4 g of crude material. This was purified by flash chromatography using the Biotage SP4 (40+M silica cartridge), eluting with a gradient of 0 to 10% MeOH in DCM to yield 5.4 g of the title compound D1.

NMR δ_{H} (CDCl₃): 1.43 (6H, s), 1.95 (1H, broad s), 3.84 (2H, s), 5.36 (1H, broad s), 7.37 (1H, dd, J=5.2, 1.6 Hz), 7.47 (1H, d, J=1.6 Hz), 7.48 (1H, broad s), 8.39 (1H, d, J=5.2 Hz).

LC-MS: MH⁺=272/274, C₁₀H₁₄BrN₃O requires 271/273.

### Example 1

### 2-Methyl-N²-({4-[4-trifluoromethyl)phenyl]-2-pyridinyl}methyl)alaninamide hydrochloride (E1)

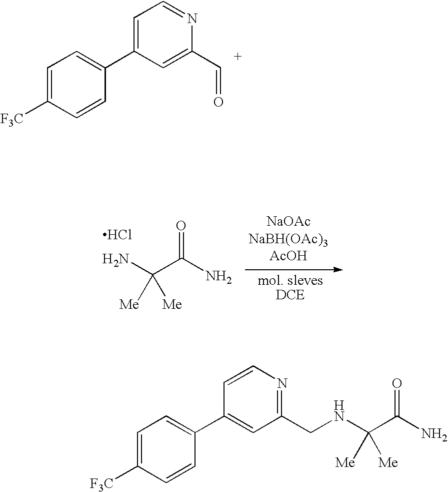

To a mixture of 2-methylalaninamide hydrochloride (D2) (224 mg, 1.63 mmol), sodium acetate (133 mg, 1.63 mmol) and molecular sieves (680 mg) in 1,2-dichloroethane (12 mL) was added 4-[4-(trifluoromethyl)phenyl]-2-pyridinecarbaldehyde (D6) (340 mg, 1.36 mmol) and the mixture was stirred under argon atmosphere at 40° C. for 1 h. NaBH(OAc)₃ (424 mg, 1.9 mmol) and AcOH (0.122 mL, 2.03 mmol) were added and the reaction mixture was stirred at room temperature overnight. TLC and LC-MS after 18 hours both showed the reaction went to completion. It was diluted with DCM (5 mL) and treated with 50% saturated aqueous NaHCO₃, gently shaken and poured into a phase separator cartridge to isolate the lower organic phase which was loaded straight into an SCX column (10 g). This was eluted with DCM, MeOH and NH₃ (2M in MeOH); the appropriate fractions were concentrated to afford 440 mg of crude material which was further purified by MDAP and the corresponding formate salt (430 mg) was isolated.

¹H-NMR (CDCl₃): δ 1.48 (6H, s), 2.35 (2H, obscured by water peak), 3.99 (2H, s), 6.0 (1H, broad s), 7.45 (1H, d), 7.49 (1H, s), 7.75 (5H, m), 8.08 (1H, s; HCO₂⁻), 8.67 (1H, d).

The compound was converted to its hydrochloride salt by treatment with HCl (1M in Et₂O, 1 eq) to yield the title compound E1 (360 mg, 71%) as a white solid.

LC-MS: MH⁺=338, C₁₇H₁₈F₃N₃O requires 337.

### Example 1a

### 2-Methyl-N²-({4-[4-(trifluoromethyl)phenyl]-2-pyridinyl}methyl)alaninamide (E1a)

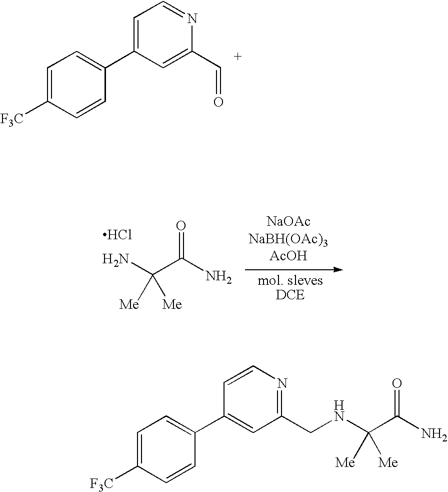

A mixture of 2-methylalaninamide hydrochloride (D2) (18.56 g, 0.134 mol), sodium acetate (11.0 g, 0.134 mol) and 4 Å molecular sieves (80 g, pellets) and 4-[4-(trifluoromethyl)phenyl]-2-pyridinecarbaldehyde (D6) (22.43 g, 0.894 mol) in 1,2-dichloroethane (700 mL) was stirred at room temperature under argon using a mechanical stirrer. After 20 h, an aliquot was removed and concentrated and NMR indicated imine formation. NaBH(OAc)₃ (28.4 g) was added followed by acetic acid (7.7 mL) and stirring was continued at room temperature for 1 h. Saturated aqueous sodium bicarbonate solution (500 mL) was added cautiously to the dark reaction mixture and the mixture was stirred at room temperature for 1 h. After standing at room temperature for 2.5 h, the mixture was filtered and the filter washed with dichloromethane and the filtrate layers were separated. The aqueous layer was extracted with dichloromethane (3×400 mL) and the combined organic layers were dried over sodium sulfate and evaporated to afford a brown solid. Chromatography on silica gel (elution with 0-100% ethyl acetate in 40-60 petroleum ether, followed by 2-10% methanol in ethyl acetate) gave the title compound as a grey solid (27.3 g). This was dissolved in ethyl acetate (500 mL) and methanol (50 mL) and stirred with charcoal (Norit SX plus, 3 g) at room temperature for 3.5 h. The mixture was filtered through Celite and the Celite was washed with ethyl acetate (3×50 mL) and methanol (1×50 mL) and the filtrate was concentrated to give 26.76 g of the title compound E1a (free base).

¹H-NMR δ_{H} (CDCl₃): 1.46 (6H, s), 2.05 (1H, broad s), 3.95 (2H, s), 5.5 (1H, broad s), 7.42 (1H, dd, J=2.0, 5.2 Hz), 7.46 (1H, s), 7.61 (1H, broad s), 7.75 (4H, m), 8.67 (1H, d, J=4.8 Hz).

### Example 1b

### 2-Methyl-N²-({4-[4-(trifluoromethyl)phenyl]-2-pyridinyl}methyl)alaninamide hydrochloride (E1)

The product from example 1a was dissolved in dichloromethane (850 mL) and treated with HCl (1 M in diethyl ether, 87.3 mL, 87.3 mmol) and the mixture was stirred at room temperature for 15 minutes. The mixture was concentrated and triturated with diethyl ether (500 mL) and the solid was collected by filtration, washed with diethyl ether (2×200 mL) and then dried in the vacuum oven at 30° C. for 20 minutes. The resulting solid was recrystallized from isopropanol (3 L) to yield a white solid. NMR indicated isopropanol was present, therefore the product was kept in a sealed vacuum desiccator under an atmosphere of water vapour overnight and then dried in a vacuum oven at 45° C. for 2 h to give the title compound (E1) as a colourless solid (25.18 g).

¹H-NMR δ_{H} (D-6 DMSO): 1.63 (6H, s), 4.34 (2H, s), 7.72 (1H, s), 7.86 (1H, m), 7.91 (1H, s), 7.94 (2H, d, J=8.4 Hz), 8.06 (3H, m), 8.76 (1H, d, J=4.8 Hz), 9.46 (2H, broad s).

LC-MS: MH⁺=338, C₁₇H₁₈F₃N₃O requires 337.

### Example 1c

### 2-Methyl-N²-({4-[4-(trifluoromethyl)phenyl]-2-pyridinyl}methyl)alaninamide (E1a)

A Sample of 2-Methyl-N²-({4-[4-(trifluoromethyl)phenyl]-2-pyridinyl}methyl)alaninamide (E1a) was prepared for solid state characterisation as follows: 2-Methyl-N²-({4-[4-(trifluoromethyl)phenyl]-2-pyridinyl}methyl)alaninamide hydrochloride (E1b) (3 g) was partitioned between saturated NaHCO₃ solution and ethyl acetate and the free base extracted into ethyl acetate. The extracts were washed with water, dried (sodium sulphate) and concentrated to give about 2.4 g of the free base (E1a). The sample was dissolved in ethyl acetate (200 mL) and methanol (20 mL) and combined with a different batch of free base (prepared analogously to Example 1a and recrystallised from ethyl acetate) to ensure homogeneity. The solvents were then removed on a rotary evaporator to give 2-Methyl-N²-({4-[4-(trifluoromethyl)phenyl]-2-pyridinyl}methyl)alaninamide free base (E1a) as a white solid. The solid was characterised as follows:

Thermal Analysis

A DSC thermogram was obtained using a TA Q1000 calorimeter. The sample was weighed into an aluminium pan, a pan lid placed on top and crimped. The experiment was conducted using a heating rate of 10° C. min⁻¹. A melting endotherm was observed at an onset temperature of 134° C.

X-Ray Powder Diffraction (XRPD)

X-ray powder diffraction (XRPD) data were acquired on a PANalytical X'Pert Pro powder diffractometer, model PW3040/60, using an XCelerator detector. The acquisition conditions were: radiation: Cu Kα, generator tension: 40 kV, generator current: 45 mA, start angle: 2.0° 2θ, end angle: 40.0° 2 θ, step size: 0.0167° 2θ, time per step, 31.75 seconds. The sample was prepared by mounting a few milligrams of sample on a Si wafer (zero background) plates, resulting in a thin layer of powder. Characteristic XRPD angles and d-spacings (determined by using Highscore software) are recorded in the Table:

| Pos. [°2 θ ± 0.1°] | d-spacing [Å] |
|---|---|
| 7.1 | 12.5 |
| 10.0 | 8.9 |
| 10.9 | 8.1 |
| 13.7 | 6.5 |
| 14.1 | 6.3 |
| 15.8 | 5.6 |
| 16.7 | 5.3 |
| 17.2 | 5.1 |
| 18.3 | 4.8 |
| 19.9 | 4.5 |
| 20.3 | 4.4 |
| 20.7 | 4.3 |
| 21.2 | 4.2 |
| 22.4 | 4.0 |
| 23.6 | 3.8 |
| 25.1 | 3.5 |
| 29.3 | 3.0 |
| 30.1 | 3.0 |

### Example 2

### N¹,2-Dimethyl-N²-({4-[4-(trifluoromethyl)phenyl]-2-pyridinyl}methyl)alaninamide hydrochloride (E2)

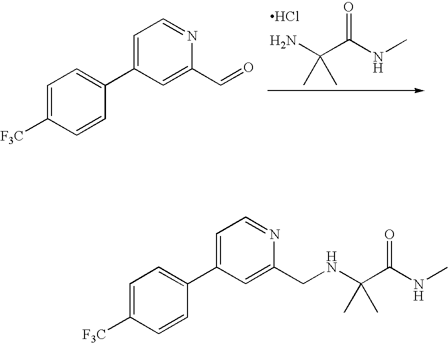

To a solution of 4-[4-(trifluoromethyl)phenyl]-2-pyridinecarbaldehyde (500 mg, 2 mmol) (D6) in DCE (20 mL) was added N¹,2-dimethylalaninamide hydrochloride (D9) (457 mg, 3 mmol), sodium acetate (246 mg, 3 mmol) and 4 Å molecular sieves (activated in the vacuum oven at 70° C. for 4 h, 2.5 g) and the resulting mixture was stirred under argon at room temperature. The imine formation was checked by ¹H-NMR and, after, 18 h NaBH(OAc)₃ (636 mg, 3 mmol) and acetic acid (0.18 mL, 3 mmol) were added. After 1.5 h the reaction mixture was filtered through a pad of Celite and concentrated in vacuo (1.04 g). The crude material was purified by flash chromatography using the Biotage SP4, eluting with a gradient of 0 to 10% MeOH in DCM to afford 698 mg of desired product E2.

LC-MS: MH⁺=352, C₁₈H₂₀F₃N₃O requires 351.

This was converted to the hydrochloride salt by addition of HCl (1 M in diethyl ether) and the resulting solid was triturated with diethyl ether, filtered and dried in vacuo to yield 375 mg of title compound (E2).

NMR δ_{H} (D₆-DMSO): 1.62 (6H, s), 2.69 (3H, d, J=4.4 Hz), 4.33 (2H, s), 7.86 (1H, dd, J=5.2, 1.6 Hz), 7.95 (2H, d, J=4 Hz), 8.06 (3H, m), 8.4 (1H, m), 8.75 (1H, d J=5.2 Hz), 9.5 (2H, broad s).

### Example 3

### N¹,N¹,2-Trimethyl-N²-({4-[4-trifluoromethyl)phenyl]-2-pyridinyl}methyl) alaninamide hydrochloride (E3)

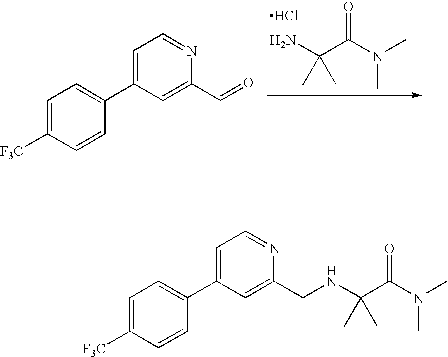

E3 was prepared via a procedure similar to that described in Example 2 starting from 4-[4-(trifluoromethyl)phenyl]-2-pyridinecarbaldehyde (500 mg, 2 mmol) (D6) and using N¹,N¹,2-Trimethylalaninamide hydrochloride (500 mg, 3 mmol) (D10). After flash chromatography 722 mg of desired product was isolated.

LC-MS: MH⁺=366, C₁₉H₂₂F₃N₃O requires 365.

This was converted to the hydrochloride salt by addition of HCl (1 M in diethyl ether) and the resulting solid was triturated with diethyl ether, filtered and dried in vacuo to yield 417 mg of title compound E3.

NMR δ_{H} (D₆-DMSO): 1.75 (6H, s), 3.05 (6H, broad s), 4.36 (2H, m), 7.86 (1H, dd, J=5.2, 2 Hz), 7.95 (2H, d, J=8.4 Hz), 8.05 (3H, m), 8.76 (1H, d, J=5.2 Hz), 9.3 (2H, broad s).

### Example 4

### N²-({4-[4-(Trifluoromethyl)phenyl]-2-pyridinyl}methyl)glycinamide hydrochloride (E4)

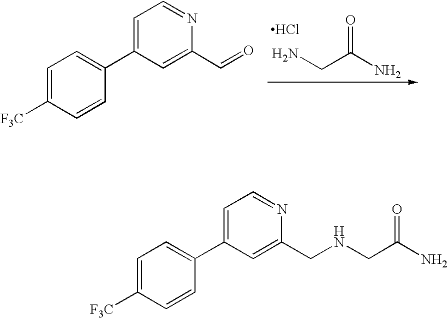

To a solution of 4-[4-(trifluoromethyl)phenyl]-2-pyridinecarbaldehyde (500 mg, 2 mmol) (D6) in DCE (20 mL) was added glycinamide hydrochloride (332 mg, 3 mmol), sodium acetate (246 mg, 3 mmol) and 4 Å molecular sieves (activated in the vacuum oven at 70° C., 2.5 g) and the resulting mixture was stirred under argon at room temperature. After 24 h, NaBH(OAc)₃ (636 mg, 3 mmol) and acetic acid (0.18 mL, 3 mmol) were added and the reaction was stirred at room temperature for a further 48 h after which it was filtered through a pad of Celite, washed with DCM and concentrated under vacuum to afford 597 mg of crude material. This was applied to a 10 g SCX cartridge, eluted with MeOH and 2M NH₃ in MeOH. The appropriate fractions were concentrated and the material was purified by flash chromatography (Biotage SP4) with a gradient of 0 to 10% MeOH in DCM (233 mg). This was further purified by MDAP and the product isolated (59 mg) was then converted to the hydrochloride salt by treatment with 1 molar equivalent of HCl (1M in diethyl ether) to yield 57.3 mg of title compound E4.

NMR δ_{H} (D₆-DMSO): 3.8 (2H, s), 4.38 (2H, s), 7.6 (1H, s), 7.85 (2H, m), 7.94 (3H, m), 8.05 (2H, d, J=8.4 Hz), 8.75 (1H, d, J=5.6 Hz), 9.36 (2H, broad s).

LC-MS: MH⁺=310, C₁₅H₁₄F₃N₃O requires 309.

### Example 5

### N²-({4-[4-Trifluoromethyl)phenyl]-2-pyridinyl}methyl)-L-alaninamide hydrochloride (E5)

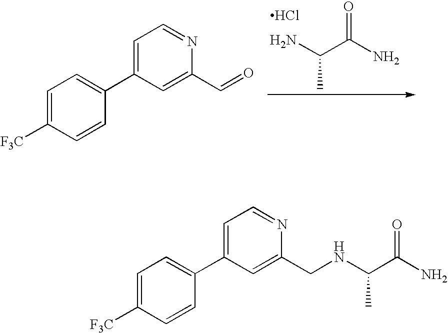

Title compound E5 was prepared via a procedure similar to that described in Example: 4 starting from 4-[4-(trifluoromethyl)phenyl]-2-pyridinecarbaldehyde (500 mg, 2 mmol) (D6) and using L-alaninamide hydrochloride (374 mg, 3 mmol). The final conversion to the hydrochloride salt yielded 58.6 mg of title compound E5. (34% e.e. by HPLC analysis on chiral column—Chiral AD 4.6 mm i.d×250 mm, 10 um—mobile phase: heptane/absolute ethanol 90/10 v/v).

NMR δ_{H} (D₆-DMSO): 1.5 (3H, d, J=6.8 Hz), 3.94 (1H, m), 4.37 (2H, m), 7.65 (1H, s), 7.85 (1H, dd, J=5.2, 2 Hz), 7.96 (3H, m), 8.02 (1H, s), 8.06 (2H, d, J=8.4 Hz), 8.75 (1H, d, J=5.6 Hz), 9.45 (2H, broad s).

LC-MS: MH⁺=324, C₁₆H₁₆F₃N₃O requires 323.

### Example 6

### N²-({4-[4-(Trifluoromethyl)phenyl]-2-pyridinyl}methyl)-D-alaninamide hydrochloride (E6)

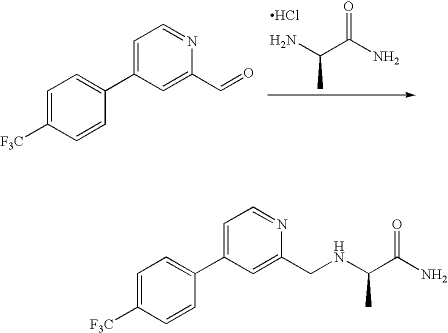

Title compound E6 was prepared via a procedure similar to that described in Example 4 starting from 4-[4-(trifluoromethyl)phenyl]-2-pyridinecarbaldehyde (500 mg, 2 mmol) (D6) and using D-alaninamide hydrochloride (374 mg, 3 mmol). The final conversion to the hydrochloride salt yielded 75.6 mg of title compound E6. (40% e.e. by HPLC analysis on chiral column—Chiral AD 4.6 mm i.d×250 mm, 10 um—mobile phase: heptane/absolute ethanol 90/10 v/v)).

NMR δ_{H} (D₆-DMSO): 1.49 (3H, d, J=6.8 Hz), 3.94 (1H, m), 4.37 (2H, m), 7.65 (1H, s), 7.85 (1H, dd, J=5.2, 2 Hz), 7.95 (3H, m), 8.00 (1H, s), 8.05 (2H, d, J=8 Hz), 8.75 (1H, d, J=5.6 Hz), 9.44 (2H, broad s).

LC-MS: MH⁺=324, C₁₆H₁₆F₃N₃O requires 323.

### Example 7

### 2-Methyl-N²-({4-[2-(trifluoromethyl)phenyl]-2-pyridinyl}methyl)alaninamide hydrochloride (E7)

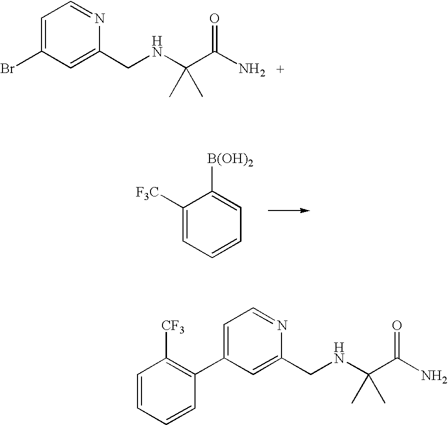

To a solution of N²-[(4-bromo-2-pyridinyl)methyl]-2-methylalaninamide (D11) (500 mg, 1.84 mmol), [2-(trifluoromethyl)phenyl]boronic acid (384 mg, 2.02 mmol) and NaHCO₃ (773 mg, 9.2 mmol) in degassed toluene/water (9/1, 10 mL) was added Pd(PPh₃)₄ (64 mg, 0.055 mmol) and the mixture was refluxed at 90° C. for 3 days. The reaction mixture was then concentrated to dryness, the residue partitioned between EtOAc (60 mL) and water (60 mL) and the organic layer was separated. The aqueous was then extracted with EtOAc and the combined organics were washed with brine and dried over MgSO₄. The crude material (590 mg) was purified by flash chromatography (Biotage SP4, 25+M silica cartridge), eluting with a gradient 0 to 10% of MeOH in DCM. The isolated material (400 mg) was further purified by MDAP and then converted to the hydrochloride salt by treatment with HCl (1M in diethyl ether). The resulting solid was then triturated with diethyl ether, filtered and dried under vacuum to yield 142 mg of title compound. E7

NMR δ_{H} (D₆-DMSO): 1.63 (6H, s), 4.33 (2H, m), 7.45 (2H, m), 7.59 (1H, s), 7.73 (2H, m), 7.82 (1H, t, J=7.2 Hz), 7.92 (2H, m), 8.72 (1H, d, J=4.8 Hz), 9.46 (2H, broad s).

LC-MS: MH⁺=338, C₁₇H₁₈F₃N₃O requires 337.

### Example 8

### 2-Methyl-N²-({4-[3-(trifluoromethyl)phenyl]-2-pyridinyl}methyl)alaninamide (E8) hydrochloride

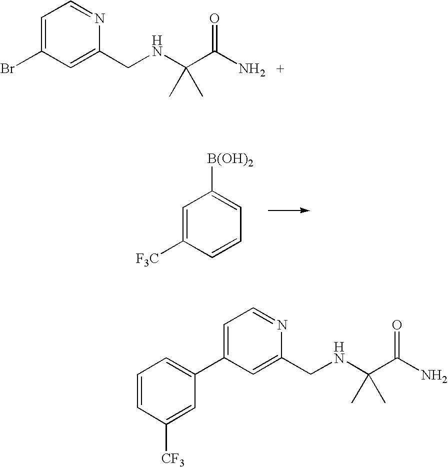

Title compound E8 was prepared via a procedure similar to that described in Example 7 starting from N²-[(4-bromo-2-pyridinyl)methyl]-2-methylalaninamide (D11) (500 mg, 1.84 mmol) and using [3-(trifluoromethyl)phenyl]boronic acid (384 mg, 2.02 mmol). The final conversion to the hydrochloride salt yielded 150 mg of title compound E8.

NMR δ_{H} (D₆-DMSO): 1.63 (6H, s), 4.35 (2H, m), 7.73 (1H, s), 7.82 (1H, m), 7.91 (3H, m), 8.1 (1H, s), 8.18 (2H, m), 8.75 (1H, d, J=5.2 Hz), 9.46 (2H, broad s).

LC-MS: MH⁺=338, C₁₇H₁₈F₃N₃O requires 337.

Biological Assays

The ability of the compounds of the invention to modulate the voltage-gated sodium channel subtype NaV 1.3 may be determined by the following assay.

Cell Biology

Stable cell lines expressing hNaᵥ1.3 channels were created by transfecting CHO cells with the pCIN5 vector (see Rees S., Coote J., Stable J., Goodson S., Harris S. & Lee M. G. (1996). A bicistronic vector for the creation of mammalian cell lines that predisposes all antibiotic resistant cells to express recombinant protein. Biotechniques, 20, 102-112) containing a neo-selectable marker with the CMV promoter, using the lipofectamine (Invitrogen) method (for full details see Chen Y H, Dale T J, Romanos M A, Whitaker W R, Xie X M, Clare J J. Cloning, distribution and functional analysis of the type III sodium channel from human brain Eur J Neurosci, 2000 December; 12, 4281-9). Cells were cultured in Iscove's modified Dulbecco's medium (Invitrogen) containing, 10% fetal bovine serum, 1% L-glutamine, 1% Penicillin-Streptomycin (Invitrogen), 1% non-essential amino acids, 2% H-T supplement and 1% G418 (Invitrogen) and maintained at 37° C. in a humidified environment containing 5% CO₂ in air. Cells were liberated from the T175 culture flask for passage and harvesting using Versene (Invitrogen).

Cell Preparation

Cells were grown to 60-95% confluence in a T75 flask. Cells were lifted by removing the growth media and incubating with 1.5 ml of warmed (37° C.) Versene (Invitrogen, 15040-066) for 6 min. Lifted cells were suspended in 10 ml of PBS (invitrogen, 14040-133). Cell suspension was then placed into a 10-ml centrifuge tube and centrifuged for 2 min at 700 rpm. After centrifugation, the supernatant was removed and the cell pellet was resuspended in 3 ml of PBS.

Electrophysiology

Currents were recorded at room temperature (21-23° C.) using the IonWorksHT planar array electrophysiology technology (Molecular Devices Corp.). Stimulation protocols and data acquisition were carried out using a microcomputer (Dell Pentium 4). In order to determine planar electrode hole resistances (Rp), a 10 mV, 160 ms potential difference was applied across each hole. These measurements were performed before cell addition. After cell addition a seal test was performed prior to antibiotic (amphotericin) circulation to achieve intracellular access. Leak subtraction was conducted in all experiments by applying a 160 ms hyperpolarizing (10 mV) prepulse 200 ms before the test pulses to measure leak conductance. Test pulses stepping from the holding potential of −90 mV to 0 mV were applied for 20 ms and repeated 10 times at a frequency of 10 Hz. In all experiments, the test pulse protocol was performed in the absence (pre-read) and presence (post-read) of a compound. Pre- and post-reads were separated by a compound addition followed by a 3-3.5 min incubation.

Solutions and Drugs

The intracellular solution contained the following (in mM): K-gluconate 100, KCl 40 mM, MgCl₂ 3.2, EGTA 3, HEPES 5, adjusted to pH 7.25. Amphotericin was prepared as 30 mg/ml stock solution and diluted to a final working concentration of 0.1 mg/ml in internal buffer solution. The external solution was Dulbecco's PBS (Invitrogen) and contained the following (in mM): CaCl₂ 0.90, KCl 2.67, K₃PO₄ 1.47, MgCl₂ 0.50, NaCl 138, Na₃PO₄ 8.10, with a pH of 7.4. Compounds were prepared in DMSO as 10 mM stock solutions and subsequent 1:3 serial dilutions performed. Finally the compounds were diluted 1:100 in external solution resulting in a final DMSO concentration of 1%.

Data Analysis

The recordings were analysed and filtered using both seal resistance (>40 MΩ) and peak current amplitude (>200 pA) in the absence of compound to eliminate unsuitable cells from further analysis. Paired comparisons between pre-compound and post-compound additions were used to determine the inhibitory effect of each compound. The concentrations of compounds required to inhibit current elicited by the 1^{st} depolarising pulse by 50% (tonic pIC50) were determined by fitting of the Hill equation to the concentration response data. In addition the use-dependent inhibitory properties of the compounds were determined by assessing the effect of compounds on the 10^{th} versus 1^{st} depolarising pulse. The ratio of the 10^{th} over 1^{st} pulse was calculated in the absence and presence of drug and the % use-dependent inhibition calculated. The data was fitted using the same equation as for the tonic pIC₅₀ and the concentration producing 15% inhibition (use-dependent pUD₁₅) calculated.

The named compounds of the invention listed hereinabove were tested in the above assay as hydrochloride salts and gave pUD₁₅≧4.8.

Rat Mustard Oil Model of Visceral Pain

All behavioural responses consistent with the presence of pain were determined following intra rectal injection of mustard oil in male Sprague Dawley rats. Typical behavioural responses consistent with the presence of pain following intra-colonic mustard oil injection include: arching, abdominal lifting, abdominal tensing, stretching, extending the rear leg (when lying down), raising and lowering the testicles, tip-toeing and writhing.

Male Sprague Dawley rats (130 g-160 g) were briefly sedated with 50% oxygen/50% carbon dioxide and 0.2 ml of 3% mustard oil injected into the colorectum, 1.5 cm from the anus. The anus was plugged with vaseline and animals placed in observational cages to which they had previously been acclimatised for 45 minutes prior to mustard oil injection. The number of visceral pain related behaviours, which consisted primarily of abdominal arching, were counted over a 25 minute period and the animals culled by cervical dislocation.

In separate studies, the effects of alosetron (0.1, 0.3 and 1.0 mg/kg n=10 per group), gabapentin (10, 30 and 100 mg/kg n=10 per group) and amitriptyline (3, 10 and 30 mg/kg n=9-10 per group) or vehicle (saline for alosetron and amitriptyline 10% 1-methyl-2-pyrolidone in saline for gabapentin n=10 per study) given sub cutaneously 15 minutes prior to injection of 3% mustard oil were examined on pain behaviour. Results are expressed as mean reduction in percentage of behaviours compared to vehicle treated animals and were statistically compared to vehicle treated animals using a one way ANOVA with Dunnett's comparison, p<0.05 considered significant.

Pre-treatment with alosetron (56%, 56% and 54% reduction in behaviours compared to vehicle treated animals at 0.1, 0.3 and 1.0 mg/kg respectively), gabapentin (28%, 50% and 69% reduction in behaviours compared to vehicle treated animals at 10, 30 and 100 mg/kg respectively) or amitriptyline (43%, 73% and 91% reduction in behaviours compared to vehicle treated animals at 3, 10 and 30 mg/kg respectively) reduced the number of behaviours observed following intra-rectal mustard oil. All measured reductions were at least significant to p<0.05, except for gabapentin at 10 mg/kg, which failed to achieve statistical significance.

The results demonstrate that intra-rectal mustard oil elicits robust and reproducible pain behaviours in the conscious rat and that pre-treatment with alosetron, gabapentin or amitriptyline attenuates this behaviour, thereby demonstrating an analgesic action of these compounds which may contribute to their known clinical efficacy in the treatment of visceral pain and/or IBS, and thus validating this model as a useful predictor of compounds likely to have an effect on visceral pain and the visceral pain associated with IBS in humans.

The effect of the compound of Example 1 (0.1, 0.3, 1.0, 3.0, 10 and 30 mg/kg, n 9-20 per dosage) or vehicle (1% methyl cellulose, n=20) given orally 60 minutes prior to injection of mustard oil (3% mustard oil, 70% ethanol in saline) were examined on pain behaviour. The total number of visceral pain related behaviours, which consisted primarily of abdominal arching, were counted over a 25 minute period and the animals culled by cervical dislocation. Behaviours were normalised as the percentage of the vehicle group mean and expressed as mean percentage reductions. Data was combined from two studies and normalised as the percentage of the vehicle group mean for each individual study and expressed as a mean percentage reduction for both studies. Percentage reduction of behaviours compared to vehicle treated animals were analysed statistically using a one way ANOVA with Dunnett's comparison, p<0.05 considered significant.

Pre-treatment with E1 reduced the number of behaviours observed following intra-rectal mustard oil (16%, 24%, 41%, 64%, 76% and 85% reduction in behaviours compared to vehicle treated animals at 0.1, 0.3, 1.0, 3.0, 10 and 30 mg/kg respectively) and was statistically significant from vehicle treated animals except at 0.1 mg/kg, which failed to achieve statistical significance.

These findings demonstrate an analgesic action for 2-Methyl-N²-({4-[4-(trifluoromethyl)phenyl]-2-pyridinyl}methyl)alaninamide (as the hydrochloride salt) in response to colorectal pain, suggesting a potential utility for this compound in the treatment of visceral pain and/or IBS.

1. 2-Methyl-N²-({4-[4-(trifluoromethyl)phenyl]-2-pyridinyl}methyl)alaninamide or a pharmaceutically acceptable salt thereof.

2. A pharmaceutical composition which comprises a compound according to claim 1 and a pharmaceutically acceptable carrier or excipient.

3. A method of treatment of irritable bowel syndrome which comprises administering to a host in need thereof an effect amount of a compound according to claim 1.

4. A method of treatment of neuropathic pain which comprises administering to a host in need thereof an effect amount of a compound according to claim 1.
